(19) United States
(12) Patent Application Publication
(10) Pub. No.: US 2009/0304768 A1
(43) Pub. Date: Dec. 10, 2009

(54) Gastro-Retentive System for the Delivery of Macromolecules

(75) Inventors: Noa Lapidot, Mevasseret Zion (IL); Michel Afargan, Ranana (IL); David Kirmayer, Ma'ale Edomim (IL); Lena Kluev, Jerusalem (IL); Marina Cohen, Jerusalem (IL); Eytan Moor, Jerusalem (IL); Nadav Navon, Rechovot (IL)

Correspondence Address: THE NATH LAW GROUP, 112 South West Street, Alexandria, VA, 22314, US

(73) Assignee: INTEC PHARMA LTD, Jerusalem (IL)

(21) Appl. No.: 12/223,965

(22) PCT Filed: Feb. 15, 2007
(86) PCT No.: PCT/IL2007/000212

§ 371 Date: Jun. 15, 2009

Related U.S. Application Data
(60) Provisional application No. 60/773,316, filed on Feb. 15, 2006.

(51) Int. Cl. A61F 2/00; A61K 38/29; B31B 1/26

(52) U.S. Cl. 424/423; 514/12; 493/243; 493/227

(57) ABSTRACT

The present invention provides a gastro-retentive delivery assembly (GRDA) comprising a folded multi-layered device comprising a macromolecule-containing compartment bordered by enveloping layers and one or more enforcing strips, the device being adapted to unfold when in a subject's stomach, whereupon unfolding, the macromolecule is released from said device via at least one aperture in an enveloping layer. The invention also provides a method for gastroretentive delivery of macromolecules via the GRDA of the invention; a method of preparing the GRDA of the invention as well as a method for treating a subject for a pathological condition, making use of the GRDA of the invention.

### FIELD OF THE INVENTION

This invention relates to oral delivery of therapeutic macromolecules.

### PRIOR ART

The following is a list of prior art which are considered to be pertinent for describing the state of the art in the field of the invention. Acknowledgement of these references herein will at times be made by indicating their number(s) from the list below within parentheses.
(1) Cordier-Bussat M, et al. Endocrinology. 138(3):l 137-44 (1997);
(2) Fuessl et al., Clin Sci (Lond). 72(2):255-7 (1987) Oral absorption of the somatostatin analogue SMS 201-995: theoretical and practical implications;
(3) Boden G, et al. Somatostatin suppresses secretin and pancreatic exocrine secretion; Science 190:163-5 (1975);
(4) Schlegel W, et al. Inhibition of cholecystokinin-pancreozymin release by somatostatin. Lancet ii:166-8 (1997);
(5) Konturek S J, et al. Studies on the inhibition of pancreatic secretion by luminal somatostatin. Am J Physiol 241:G109-15 (1981);
(6) Sarfati P and Morisset J. Regulation of pancreatic enzyme secretion in conscious rats by intraluminal somatostatin: Mechanism of action. Endocrinology 124:2406-14 (1989);
(7) Homik et al. in U.S. Pat. No. 6,930,088;
(8) Homik et al. in U.S. Pat. No. 6,355,613;
(9) Homik et al. in U.S. Pat. No. 6,051,554;
(10) Byun et al. in U.S. Pat. No. 6,656,922;
(11) Friedman et al. U.S. Pat. No. 6,685,962.

### BACKGROUND OF THE INVENTION

The efficacious delivery of macromolecules to their site of action in the body requires addressing of some inherent obstacles. Major obstacles are the commonly found instability of macromolecules in various organs and tissues and the difficulty in absorption of macromolecules across membrane barriers at the root of administration such as the gastric lumen for oral intake or the lining of the epithet if pulmonary route is used, or the skin for topical administration. Another issue with delivery of macromolecules is the need to provide some of these active compounds over an extended period of time, at a controlled level.

Typically, absorption or activity sites of biomacromolecules are located in the upper part of the GI tract, namely in the stomach, the duodenum, jejunum, illeum and through Peyers patches expressed along the gastrointestinal tract. For example, some gastrointestinal peptide hormones have biological activity in the gastrointestinal tract and are naturally secreted locally (i.e., gastrin which is secreted from the stomach G-cells, somatostatin secreted from the stomach delta cells, cholecystokinin-pancreozymin secreted from the duodenum (1). Peptones stimulate cholecystokinin secretion and gene transcription in the intestinal cell line STC-1. As such, these peptides or their synthetic, metabolic stable analogues may be utilized for therapeutic purposes, providing they could be effectively administered. Moreover, some of these peptides have systemic effects, but are only absorbed in the small intestine and not in the colon (2).

One way to overcome the instability of macromolecules in the gastrointestinal tract is to administer these molecules intraluminaly (into the gastrointestinal lumen) in a continuous manner (i.e., infusion). For example, there have been reports showing that an intraluminal continuous administration by infusion of native somatostatin results in significant biological activities that are common for this peptide hormone (3-5). Other reports demonstrated the inhibitory effect of the peptide hormone somatostatin found in gastric fluids on gastric pH and gastric secretions (6).

Alternatively, native hormones such as somatostatin, cholecystokinin (CCK), Thyroid Releasing Hormone (TRH), secretin and others may be administered intra gastrically or into the intestine

Many of these studies dose the peptides to the intestine by gavage. This, however, is not a feasible way of treatment when continuous delivery of the molecule is required for its action.

During the last decades numerous efforts were focused on chemical approaches to improve the metabolic stability of biologically active macromolecules. It should be emphasized that all the types macromolecules discussed here are also part of normal diet and as such they are also considered as nutrients, hence their natural instability (i.e., hydrolysis, degradation) occurs in a more extensive manner following oral intake. The biological conditions that pertain in the gastrointestinal tract such as the acidic pH of the stomach and the wide range of metabolic enzymes active in the gut enable the physiological process of food digestion. For example gastric and intestinal enzymes (i.e., pepsin, trypsin and chymotrypsin) metabolize polypeptides and peptides to amino acids ready for absorption; gastric and intestinal lipases metabolized lipids; the enzyme family of amylases degrades polysaccharides. Several strategies to overcome the inherent instability have been developed. For example, it has been proven that cyclic analogues of peptides have stability greater by orders of magnitude compared to the natural ones (7-10). Molecular analogues designed for improved stability indeed have been shown to have similar activity to the native molecule but have a much longer life time in the body (i.e. somatostatin analogues). Other strategies to improve oral delivery include mixing the peptides with protease inhibitors, such as aprotinin, soybean trypsin inhibitor and antibiotics such as bacitracin, in an attempt to limit degradation of the administered therapeutic agent. Unfortunately these protease inhibitors are not selective, and endogenous proteins are also inhibited.

Various strategies are being used in attempts to improve absorption of peptides in the GIT. These strategies include incorporation of absorption enhancers, such as the salicylates, lipid-bile salt-mixed micelles, glycerides, and acylcarnitines, but these frequently are found to cause serious local toxicity problems, such as local irritation and toxicity, complete abrasion of the epithelial layer and inflammation of tissue.

To date, the major route of administration of macromolecules is either through parenteral or intravenous injections. For example, the hormone insulin, used in the treatment of diabetes, has been administered for many years through subcutaneous injections only, in spite of enormous scientific and technological efforts to develop alternative routes; Similarly, erythropeitin and monoclonal antibodies are delivered by injection, or by a long acting release (LAR) formulation—an injected depot that reduces the frequency of injections to once every 28 days.

Nonetheless, oral intake is a preferred mode of administration for many drugs for ease of use. This is true for drugs that have to be absorbed systemically, and even more so for macromolecules that should act inside the gastrointestinal tract, for example satiety controlling hormones that have local activity (as well as systemic activity) and locally acting enzymes such as gastric lipase that is used in the treatment of cystic fibrosis. Thus, means to overcome the hurdles to oral formulation of macromolecules are sought after. Such means should address the instability issues, the absorption issues and the kinetics of release of the drug to its absorption site in the gastrointestinal tract or the site of action in the GI tract.

Devices that can be retained in the stomach for periods of 3 to 24 hours and release a drug therefrom in a controlled manner are described (11).

### SUMMARY OF THE INVENTION

The present invention provides a gastro-retentive delivery assembly (GRDA), comprising a folded multi-layered device comprising a macromolecule-containing compartment bordered by enveloping layers, and comprising one or more enforcing strips, the device being adapted to unfold to when in said subject's stomach, whereupon unfolding, the macromolecule is released from said device via at least one aperture in an enveloping layer.

The invention also provides a method for delivery of macromolecules to a subject's stomach, the method comprising administration to said subject of the GRDA of the invention. The delivery of the GRDA is preferably oral delivery.

Yet further, the invention provides a method of preparing a GRDA for delivery of macromolecules comprising: (i) assembling a multi-layered device comprising a macromolecule-containing compartment bordered by enveloping layers, at least one of said enveloping layers is made of a film comprising at least one aperture or a polymeric composition comprising a material which dissolves upon contact with gastric fluid to form at least one aperture and one or more enforcing strips, the device being adapted to unfold when in a subject's stomach, whereupon unfolding, the macromolecule is released from said device via the at least one aperture; (ii) folding said device; and (iii) introducing or combining the folded device with a delivery system.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a graph showing the release of α-Melatonin Stimulating Hormone (MSH) into KCl/HCl buffer pH=2.2 of GRDA 1 (-□-) and GRDA 2 (-♦-).

FIG. 2 is a graph showing the release of α-MSH into KCl/HCl buffer pH=2.2 of GRDA 3 having apertures in the enveloping layer of 1.5 mm (-♦-) or 0.7 mm (-□-) in diameter.

FIG. 3 is a graph showing the release of Parathyroid Hormone (PTH) fragment-1-34 of GRDA 7 into KCl/HCl buffer pH=2.2.

FIG. 4 is a graph showing the stability of PTH 1-34 of GRDA 7 in various buffer solutions.

### DETAILED DESCRIPTION OF EXEMPLARY EMBODIMENTS

Macromolecules (biomacromolecules), being large, three dimensional structures may require a different strategy when designing a delivery system for them. It has now become evident that formulations provided for low molecular weight drugs, such as those incorporated in the gastro-retentive delivery formulation (GRDF) described in U.S. Pat. No. 6,685,962, may not be suitable for the delivery of macromolecules. Specifically, in addition to their size limitation, macromolecules, as compared to low molecular weight compounds, are typically more sensitive to various chemical reagents, temperature conditions, oxidizing agents etc., all of which may affect the delivery and functionality of the macromolecule.

It is therefore desired to provide a GRDA, preferably in a delivery system, for oral intake of biologically functional and active macromolecules.

In accordance with a first of its aspects, the present invention provides a GRDA for delivery of macromolecules to a subject, comprising a folded multi-layered device comprising a macromolecule-containing compartment bordered by enveloping layers and comprising one or more enforcing strips, the device prior to folding being essentially planar, the delivery system being adapted to unfold when is said subject's stomach, whereupon unfolding the macromolecules are released from the device via an aperture in an enveloping layer.

The term “macromolecule” as used herein denotes any natural, synthetic or semi-synthetic substance having a molecular weight of at minimum about 1,800 Da, preferably at least about 2,000 Da, more preferably, at least about 3,000 Da and most preferably at least about 4,000 Da. The macromolecule may be a carbohydrate, a nucleic acid molecule, an amino acid molecule, a lipid, a vitamin or vitamin analogue or any other organic molecule, having a biological functionality and activity. In other words, the macromolecule is any large molecule (i.e. MW greater than about 1,800 Da, preferably about 2,000 Da, more preferably than about 3,000 Da and most preferably greater than about 4,000 Da) which may be utilized as a therapeutic agent.

The term “carbohydrate” denotes any saccharide-containing compound including, without being limited thereto, oligosaccharides and polysaccharides as well as substances derived from mono-, oligo- or polysaccharides by reduction of the carbonyl group (alditols), by oxidation of one or more terminal groups to carboxylic acids, or by replacement of one or more hydroxy group(s) by a hydrogen atom, an amino group, a thiol group or similar heteroatomic groups. It also includes derivatives of such compounds, such as conjugates with a different type of compound, e.g. lipopolysaccharide.

The term “oligosaccharide” denotes any saccharide containing compound in which monosaccharide units (between 2 to 10) are joined by glycosidic linkages. According to the number of units, they are called disaccharides, trisaccharides, tetrasaccharides, pentasaccharides etc. Polysaccharide denotes a saccharide-containing a large number of monosaccharide (glycose) residues, typically more than 10 units, joined to each other by glycosidic linkages. Oligosaccharide analogues, which also form part of the invention, are saccharide containing compounds in which the linkage between the units are of a type other than glycosidic linkages, as known to those versed in the art.

According to one embodiment, the oligosaccharides include, without being limited thereto heparin, heparin derivatives i.e. heparin covalently bonded to a hydrophobic agent such as bile acids, sterols, and alkanoic acids, and mixtures thereof as described, for example in U.S. Pat. No. 6,656,922, Byun, et al. incorporated herein by reference] as well as modifications with a hydrophilic group (hydrophobized heparin) or with a lipid (amphiphilic heparin), as appreciated by those versed in the art.

The term “amino acid molecule” denotes any compound comprising two or more amino acid residues joined together, preferably by a peptide bond to form a peptide, a protein, a polypeptide as well as peptidomimetic molecules. The amino acid residue may be any one of the 20 conventional, naturally occurring amino acids, as well as stereoisomers (e.g., D-amino acids) of the twenty conventional amino acids, unnatural amino acids such as α,α-disubstituted amino acids, N-alkyl amino acids, lactic acid, and other unconventional amino acids known to those versed in the art. Examples of unconventional amino acids include: 4-hydroxyproline, γ-carboxy-glutamate, ε-N, N, N-trimethyllysine, ε-N-acetyllysine, O-phosphoserine, N-acetylserine, N-formylmethionine, 3-methylhistidine, 5-hydroxylysine, ω-N-methyllarginine, and other similar amino acids and imino acids (e.g., 4-hydroxyproline), etc. as known to those versed in the art.

When including a non-naturally occurring amino acid residue the amino acid molecule may be referred to by the term “peptidomimetic”. Peptidomimetics are often used to inhibit degradation of the amino acid molecules by enzymatic or other degradative processes and can be produced by organic synthetic techniques. Examples of suitable peptidomimetics include the above mentioned D-amino acids of the corresponding L amino acids, tetrazol (Zabrocki et al., J. Am. Chem. Soc. 110:5875-5880 (1988)); isosteres of amide bonds (Jones et al., Tetrahedron Lett. 29: 3853-3856 (1988)); LL-3-amino-2-propenidone-6-carboxylic acid (LL-Acp) (Kemp et al., J. Org. Chem. 50:5834-5838 (1985)). Similar analogs are shown in Kemp et al., Tetrahedron Lett. 29:5081-5082 (1988) as well as Kemp et al., Tetrahedron Lett. 29:5057-5060 (1988), Kemp et al., Tetrahedron Lett. 29:4935-4938 (1988) and Kemp et al., J. Org. Chem. 54:109-115 (1987). Other suitable peptidomimetics are shown in Nagai and Sato, Tetrahedron Lett. 26:647-650 (1985); Di Maio et al., J. Chem. Soc. Perkin Trans., 1687 (1985); Kahn et al., Tetrahedron Lett. 30:2317 (1989); Olson et al., J. Am. Chem. Soc. 112:323-333 (1990); Garvey et al., J. Org. Chem. 56:436 (1990). Further suitable peptidomimetics include hydroxy-1,2,3,4-tetrahydroisoquinoline-3-carboxylate (Miyake et al., J. Takeda Res. Labs 43:53-76 (1989)); 1,2,3,4-tetrahydro-isoquinoline-3-carboxylate (Kazmierski et al., J. Am. Chem. Soc. 133:2275-2283 (1991)); histidine isoquinolone carboxylic acid (HIC) (Zechel et al., Int. J. Pep. Protein Res. 43 (1991)); (2S, 3S)-methyl-phenylalanine, (2S, 3R)-methyl-phenylalanine, (2R, 3S)-methyl-phenylalanine and (2R, 3R)-methyl-phenylalanine (Kazmierski and Hruby, Tetrahedron Lett. (1991)).

According to one embodiment, the term “amino acid molecule” encompasses peptide therapeutics, such as, without being limited thereto, gastrin-releasing peptide, defensins (α-defensins and β-defensins), all of which have been shown to be therapeutically active with respect to conditions of the GI tract.

According to another embodiment and as also mentioned above, the term “amino acid molecule” encompasses peptidomimetic molecules, such as, without being limited there to, metabolic stable somatostatin and galanin analogs having a molecular weight as defined in the context of the present invention. One example includes somatostatin-28 and its analogs.

According to yet another embodiment, the term “amino acid molecule” encompasses enzymes, such as gastric enzymes, i.e. enzymes which are active in the gastrointestinal tract, including, without being limited thereto, pepsin, gastric and pancreatic lipase, elastase, amylase, α- and β-glycosidase, trypsin, lactase and chemotrypsin. Administration of gastric lipases may be of therapeutic benefit in treating numerous conditions including, for example, cystic fibrosis (when the enzyme is locally delivered to the stomach), chronic pancreatitis (CP), post surgery or cancer conditions, low level or lack of lipase, due to exocrine pancreatic insufficiency (EPI), which may cause an inability to digest food lipids, and to lead to steatorrhea, (excess of fats in faeces).

In accordance with yet another embodiment, the amino acid molecule is an antigen or a fragment of an antigen that can induce GIT mucosal immunity. A non-limiting example of an antigen which may be utilized in accordance with the invention is Hepatitis B surface Antigen (HBsAg) Hepatitis B core Antigen (HBcAg), recombinant cholera toxin B-subunit (rCTB) against Helicobacter pylori, HIV-P1 peptide against HIV.

Other biologically functional and active amino acid molecules may include hormones, such as, without being limited thereto, somatostatin-28, cholecystokinin, gastrin, secretin, leptin, gherlin, obestatin, neuropeptide Y-NPY, peptide—YY PYY₃₋₃₆, galanin, glucagons, glucagons like peptide, pancreatic polypeptide, oxyntomodulin, Vasoactive Intestinal Peptide (VIP), glucose-dependent insulinotropic polypeptide—GIP, motilin (all the aforementioned being gut hormones), insulin, insulin growth factor 1, luteinizing hormone (LH), follicle stimulating hormone (FSH), prolactin, adrenocorticotrophic hormone (ACTH) growth hormone, atrial-natriuretic peptide (ANP) or atrial natriuretic factor (ANF), paratyroid hormone (PTH), calcitonin, endothylin.

The term “nucleic acid molecule” denotes any compound comprising two or more nucleotide residues, comprising the conventional, naturally occurring nucleotides (usually adenine, cytosine, guanine, thymine, uracil), nucleoside residues as well as synthetic or semi-synthetic analogous of nucleotides and nucleosides as known in the art.

According to one embodiment, the biologically functional nucleic acid molecule includes, without being limited thereto, synthetic immunostimulatory nucleic acid sequences (ISS-ODN also known as CpG-ODNs). ISS-ODNs have been shown to display Th1-biassed immunoadjuvant activity upon co-administration with a variety of antigens [Kedar E. et al. Vaccine. 20(27-28):3342-54 (2002)].

According to another embodiment, the nucleic acid molecule is a gene, a gene fragment or a gene (or gene fragment) containing molecule suitable for gene therapy The nucleic acid molecule may be translocated into a target cell according to techniques known to those versed in the art, e.g. by the use of suitable vectors, for correcting defective genes responsible for disease development. Correction of a faulted gene may be through the insertion of a rectifying nucleic acid into a genome together with suitable carriers for enabling their insertion into the cell and/or genome as know to those skilled in gene therapy.

The term “lipid” denotes any compound that is soluble in non-polar solvents, including saponifiable lipids, such as glycerides (fats and oils) and phospholipids, as well as non-saponifiable lipids, principally steroids (e.g. hormones).

According to one embodiment, the biologically functional lipid is selected from phospholipids, glycolipids, glycerides, triglycerides, wax, terpenes, terpenoids, steroids, prostaglandins etc.

In the context of the present invention the term “macromolecule” also includes any combination (either by chemical bonding or by physical association to form e.g. conjugate, assembly or complex) of the above, including, without being limited thereto, lipoproteins, nucleoproteins, lipopolysaccharides, glycoproteins, pegylated proteins or polypeptides and the like. One example of a conjugate may include the association (either covalent linkage or by physical association such as entrapment in, adsorption on, aggregation or complexing with etc.) of the macromolecule with to an adjuvant (e.g. immuno-stimulating agent).

In the context of the present invention, the macromolecule may be an active principle, or a precursor of an active principle, e.g. a pro-drug having a molecular weight as defined.

In the context of the present invention, the macromolecule may be formulated with its competitive analog, as known to those versed in the art (e.g. gastrin and gastrin analog). One advantage of combining a macromolecule with its competitive is to “mask” the active principle from degradation by enzymes.

In the context of the invention, the macromolecules may also include a combination of macromolecules as defined; the macromolecules, for example, may be combined with other macromolecules acting as absorption enhancers for the former; or the macromolecules may be formulated with small molecular weight substances (MW 1,800), the macromolecules acting as carriers for these substances.

In accordance with one preferred embodiment, the macromolecules are essentially stable (as appreciated by those versed in the art) in an acidic pH, e.g. that of the gastric medium.

In accordance with another preferred embodiment, the macromolecule is characterized in that they it compatible with, at least the material forming the enveloping layer. In other words, that the material forming the enveloping layer is essentially inert with respect to the macromolecule, thereby, essentially no association between the macromolecule and the enveloping layer occurs following unfolding and wetting of the GRDA.

In accordance with yet another embodiment, the macromolecule is characterized in that has no more than about 15% adsorption via the GI tract, when administered orally without the GRDA of the invention.

In accordance with yet another embodiment, the macromolecule is characterized in that it has a therapeutic effect under fed as well as fasted conditions when administered within the GRDA of the invention.

The term “biologically functional/active macromolecule” or “active principle” denotes any macromolecule (natural, synthetic or semi-synthetic) exhibiting a measurable therapeutic and/or biochemical effect when brought into contact with a target cell, tissue or organ, such as the activation, enhancement or inhibition of a biochemical cascade. The effect preferably leads to an improvement in the medical state of the subject treated with the macromolecule and thus is referred to herein at times by the term “therapeutic effect” as further defined hereinafter.

The GRDA is administered to a subject in need, by active or passive swallowing. Once it is wetted in the gastric lumen (by the gastric fluids), the delivery device comprising the macromolecule is released from the delivery system (e.g. a capsule, as further discussed hereinbelow) and unfolds to a configuration which enables the retention of the unfolded device in the stomach for a time sufficient for achieving a measurable therapeutic effect.

As used herein, the term “folded” denotes any manner known in the art to reduce an effective projection surface: volume ratio of a generally planar layer, and includes, without being limited thereto, one or more of folding about fold lines, bending, twisting, wrapping, winding, crimping and the like.

In some preferred embodiments, the delivery device is folded parallel to the width of the unfolded device and designed to have folds which are symmetric mirror images about a first axis. This manner of folding provides an accordion-like configuration for the device.

According to another embodiment, the folded form of the device has folds of increasingly smaller amplitudes upon extending away from the first axis so as to form a partially rounded cross section and to allow the folded form to easily be inserted into a delivery system. In accordance with one embodiment, the delivery system is an essentially cylindrical container. The delivery system is further discussed hereinbelow.

According to yet another embodiment, the folded form of the device has folds of increasingly larger amplitudes upon extending away from one end of the first axis to its other end, so as to form a fan-like configuration.

In the context of the invention, the term “unfolded” denotes an essentially and generally planar configuration of the device. The term “essentially planar” or “generally planar” denotes a fully planar as well as wiggly or wavy shape of the device. Unfolding denotes any form of expansion of the device, which may result form unwinding, unrolling, inflating, swelling, and the like. Following expansion in the stomach, the unfolded and essentially planar device maintains its firmness due to its unique characteristics, as exemplified below.

The desired configuration of the multi-layered device, once unfolded, may be achieved by the incorporation of an enforcing polymeric composition, i.e. the enforcing strips) having a mechanical strength forcing, after oral intake and wetting by gastric fluids, the opening of the folded device to an essentially planar configuration.

According to one embodiment, the enforcing polymeric strips are continuous or non-continuous. For example, the strips may define a continuous or non-continuous frame with an outer rim overlapping the outer rim of the enveloping layer. The continuous or non-continuous frame may be either affixed or attached to the enveloping layer or integrally formed with the enveloping layer. Preferably, the device comprises two enveloping layers sandwiching the macromolecule containing internal space.

According one embodiment, the enforcing strips are in the form of a continuous or non-continuous frame, and have inner boundaries which at least partially enclose the macromolecule containing compartment.

To provide the desired enforcement in its unfolded state, it is preferable that the enforcing strips comprises polymeric composition comprising an enteric or non-enteric polymer, insoluble in gastric content or a combination of enteric and non-enteric insoluble polymers. Pharmaceutically acceptable enteric and non-enteric insoluble polymers are known and readily available to those versed in the art.

An enteric polymer is preferably such that it is substantially insoluble at a pH of less than 5.5. Non-limiting examples of enteric polymers applicable with respect to the invention include, shellac, cellacefate, hypromelose phthalate, hydroxypropyl methylcellulose acetate succinate, zein, polyvinyl acetate phthalate, aliginic acid and its salts, carboxymethyl cellulose and its salts, methylmethacrylate-methacrylic acid copolymers, including ethyl acrylate copolymers (polymethacrylates), or substantially insoluble (at pH of less than 5.5) derivatives of any one of the above as well as any appropriate combination of two or more of the above.

Non-limiting examples of non-enteric polymers applicable with respect to the invention include ethylcellulose; cellulose acetate; a copolymer of acrylic acid and methacrylic acid esters, having of from about 5% to about 10% functional quaternary ammonium groups; a polyethylene; a polyamide; a polyester; polyvinylchloride; cellulose acetate butyrate, polyvinyl acetate; and a combination of any two or more thereof.

In addition to the above mentioned polymeric composition, the enforcement may be achieved by combining in the polymeric composition an insoluble polymer with a further polymer, soluble in gastric content. The soluble polymer may be entrapped in the insoluble polymer or it may be modified, for example by cross-linked with the insoluble polymer, in such way that it does not exude from the polymer composition, unless disintegrating of the whole enforcing polymeric composition.

Non-limiting list of soluble polymers which may be combined with the insoluble polymer, forming together the enforcing polymeric composition, comprises proteins, polysaccharides, including gums (e.g. carrageenans, ceratonia, acacia, tragacanth, guar gum and xanthan gum), gelatine, chitosan, polydextrose, cellulose derivatives, such as hydroxypropyl cellulose, hypromelose, hydroxyethyl methyl cellulose, methyl cellulose; polyethylene oxides, polyvinyl alcohols, povidones (PVP), methacrylic acid copolymer with dimethyl amino ethyl methacrylate (Eudragit E™), propylene glycol alginate, polyethylene glycols, poloxamers, and soluble derivatives of any one of the above as well as any combination of two or more thereof.

As disclosed herein, the enforcing polymeric strips preferably provide the mechanical properties and strength of the device once unfolded. The enforcing strips are preferably characterized by a flexural strength and both between 25 and 200 kgf/mm² after immersion in simulated gastric fluid.

The term “macromolecule-containing compartment” as used herein denotes one of the following:

(i) a void bordered by a combination of continuous or non-continuous enforcing strips with enveloping layers, where the macromolecules are either contained freely in the void (e.g. in the form of dispersed dry powder or any other form of particulate matter) or adsorbed onto an internal surface of the enforcing strip and/or enveloping layer(s). When the macromolecules are in the form of particulate matter, the latter may include nano- or microspheres, nano- or microcapsules accommodating the macromolecules (by embedding, entrapping or having the macromolecules affixed to the particles' outer surface). The particulate matter may also include aggregates as well as colloids of the macromolecules.

(ii) a matrix (e.g. polymeric sheet) accommodating the macromolecule by embedment, entrapment, encapsulation, attachment, or absorbance of the macromolecule, respectively, within or to the matrix. The matrix may comprise one or more polymers, including, without being limited thereto, polymers soluble in gastric fluids, polymers insoluble in gastric fluids, as well as a combination of at least one such soluble polymer and at least one such insoluble polymer, all of which being as defined above.

The enforcing strip(s) is in association with the macromolecule-containing compartment and with the enveloping layers bordering the compartment.

The term “association” refers to any means of contact between the enforcing strip(s), the enveloping layers and macromolecule-containing compartment, including, without being limited thereto physical adjacency, physical bonding, chemical bonding etc., as well as any means of contact between the enforcing strip and enveloping layer, including, without being limited thereto, adhering, affixing of attaching, or, alternatively, the enforcing strip may form an integral part of at least part of the enveloping layer.

In accordance with one embodiment, the enforcing strips, the enveloping layers and the macromolecule containing compartment form together a laminated assembly.

The enveloping layers enclose the macromolecule containing compartment from two faces of the compartment, thereby protecting the macromolecules from gastric environment.

The enveloping layers comprise one or more polymers selected from polymers soluble in gastric content, polymers insoluble in gastric content, and combinations of any two or more thereof. Specifically, the enveloping layers comprise at least one polymer that forms a film or sheet that is permeable to the gastric fluid. Unless manipulated as described below, the polymer is selected such that, upon assembly with the enforcing strips to and macromolecule containing compartment, they form outer layers that are impermeable to macromolecules, thereby facilitating the existence of a separate compartment/area at an internal space of the device containing the macromolecules Hence, although the device releases the active macromolecules over prolonged periods, the macromolecules are protected until the GRDA is wetted by gastric fluid and they are actually released from the GRDA.

According to one embodiment, the enveloping layer is comprised of a mixture of a soluble polymer and an enteric polymer. According to another embodiment, the enveloping layer comprises a cross-linked soluble polymer, e.g. an enzymatically hydrolyzed cross-linked gelatin and a derivative thereof. A non-limiting example includes gelatin cross-linked with glutaraldehyde.

By another, non-limiting example, the enveloping layer composition comprises polyvinyl alcohol film, cross-linked with glutaraldehyde. Alternatively, said polyvinyl alcohol film could be subjected to one or more freeze-thaw cycles to induce crystallization.

Yet, in accordance with another non-limiting example, the enveloping layer composition comprises polyethylene oxide film, cross-linked by gamma irradiation.

In yet another non-limiting example the enveloping layer composition comprises polydimethyl siloxane and its derivatives.

The delivery system incorporating therein the folded multi-layered device may be any pharmaceutically acceptable orally delivered container, as known in the art of pharmaceutical delivery vehicles. The container may be, without being limited thereto, a capsule (soft or solid) containing the folded device, an elongated tube, a ring or a thread (one or more) surrounding the folded device (e.g. a polymeric thread wrapping the device in a manner resembling a cocoon), a polymeric coating,, a polymer or gel matrix embedding the folded device and the like. The single or multi layered device may be released from the delivery system as a result of the dissolution or breakdown of the delivery system when wetted by gastric fluids. A preferred container in accordance with the invention is a hard gelatin capsule, e.g. E00 hard gelatin capsule.

Upon release from the delivery system, the device is wetted and unfolds. Macromolecules are released from the multi-layered device via apertures in the enveloping layer. The apertures may be provided a priori, e.g. by mechanical puncturing of the enveloping layer prior to assembly of the different device's layers (e.g. by the manual use of commercially available punchers, e.g. circular puncher); or as a result of in situ degradation/dissolution of one or more components of the enveloping layer once brought in contact with gastric fluids which result in the formation of gaps/voids/channels in the composition forming the enveloping layer. Alternatively, the apertures may be formed during the preparation of the enveloping layer. One example is the use of a freeze drying and cross-linking technique to form porous scaffolds [Hae-Won Kim et al. J Biomed Mater Res A.; 72(2):136-45 (2005)].

The aperture containing enveloping layer may be produced according to known methods for the production of membranes with controlled porosity (for example, Handbook of Industrial Membrane Technology, MC Porter (Ed.), Noyes Publications, NJ, 1990; Membrane Formation and Modification, I Pinnau and B D Freeman (Eds.) ACS Symposium Series, ACS 2000), utilizing physical methods such as phase separation (for example U.S. Pat. No. 5,091,086, U.S. Pat. No. 4,954,381), controlled solvent boundary (U.S. Pat. No. 4,898,698), rapid de-gassing, controlled purging of gas (U.S. Pat. No. 5,958,451) or other known technologies. The pores in such sheets forming the enveloping layers are regarded as apertures, in the context of the present invention.

Apertures of controlled size may also be formed in the enveloping layer by means of a laser (for example, Nano and Micro Engineered Membrane Technology, Cjm Van Rijn (Ed.) Membrane Science and Technology Series, 10, Elsevier, Oxford, UK, 2004). In situ formation of apertures may be achieved by incorporating in the enveloping layer polymers that are soluble in gastric environment and have low miscibility with the membrane forming polymer. Alternatively, apertures may be formed in situ be incorporation of small acid soluble salts such as CaCO₃ or by incorporation of particles that are acid soluble in the enveloping layer.

In the context of the present invention, “polymers soluble in gastric fluids” (or in short, “gastric soluble”), include a polymer that forms a hydrogel or dissolved in gastric fluids at 37° C. In this connection, the term “hydrogel-forming polymer” denotes a polymer or a mixture of polymers that once in gastric fluid, absorb an amount of gastric fluid which results in the formation of a gel phase within the GRDA.

According to one embodiment, the polymer soluble in gastric content comprises one or more polymers selected from a hydrogel-forming polymer, a non-hydrogel polymer, or any combination thereof.

Non-limiting examples of gastric soluble hydrogel-forming polymer comprise proteins, polysaccharides, including gums (e.g. carrageenans, ceratonia, acacia, tragacanth, guar gum and xanthan gum), gelatine, chitosan, polydextrose, cellulose derivatives, such as high molecular weight grades of hydroxypropyl cellulose, hypromelose, hydroxyethyl methyl cellulose, hydroxyethyl cellulose, methyl cellulose, polyethylene oxides, polyvinyl alcohol and derivatives of any one of the above which are soluble in gastric fluid as well as any combination of two or more thereof, the combination also being soluble in gastric fluid.

Non-limiting examples of gastric soluble, non-hydrogel-forming polymer comprise povidones (PVP), vinyl acetate copolymers (copovidone), methacrylic acid copolymer with dimethyl amino ethyl methacrylate (Eudragit E™), low molecular weight grades of hydroxypropyl cellulose, propylene glycol, alginate, polyethylene glycols, poloxamers and soluble derivatives of any one of the above as well as any combination of two or more thereof. These soluble polymers can be further cross-linked, either with use of appropriate chemical cross-linking agent, or by physical cross-linking techniques, or via exposure to gamma radiation, to control their mechanical properties and behavior upon contact with simulated and natural gastric fluid.

As used herein, the term “insoluble polymer” denotes a polymer that when immersed in gastric fluids at 37° C. it does not lose more than 10% of its dry weight into the medium by dissolution. Consequently, films and layers comprising one or more insoluble polymers will preserve their shape in gastric fluid for at least 2 hours. A non-limiting list of polymers that are insoluble (non-degradable) comprises any polymer selected from a pharmaceutically acceptable enteric polymer, a pharmaceutically acceptable non-enteric polymer, or any combination thereof. An example for a non-degradable polymer includes polyvinyl acetate, without being limited thereto.

It is preferable that at least one enveloping layer comprise a plurality (i.e. two or more) apertures. According to the invention the term “plurality of apertures” denotes two, preferably more, holes of any shape in the enveloping layer. The apertures may be, e.g., circular, oval, and star-like shaped; the apertures may have a fixed dimension or have various dimensions; they may be randomly distributed in the layer or have a specific distribution pattern, e.g. in radially, longitudinally and/or diagonally arranged lines or in a sprinkle-like arrangement. The shape and dimension of each of the apertures may be the same or may vary within a single layer.

The release of the macromolecule has an infusion-like release profile, i.e. continuous slow release. Preferably, the release is a controlled release. The term “controlled release” or “controlled rate” equally refers to any one of sustained (i.e. delayed release), slow release, prolonged release etc., of the macromolecule from the delivery device into its surrounding. This enables the continuous delivery of relatively small amounts of the macromolecule to the surrounding (the released amount being dictated by the specific selection and design of the different components of the device) for a time period sufficient to achieve a therapeutic effect. The time period is preferably equivalent to the retention time of the gastroretentive device in the stomach.

In this context, the term “gastro-retentive” or “gastro-retentivity” denotes the maintenance or withholding of the macromolecule in the GI tract, for a time period longer than the time it would have been retained in the stomach when delivered in a free form or within a gastro-intestinal delivery vehicle which is not considered gastro-retentive. Gastro-retentivity may be characterized by retention in the stomach for a period that is longer than the normal emptying time from the stomach, i.e. longer than about 2 hours, particularly longer than about 3 hours and usually 4, 6, 8 or 10 hours. Gastroretentivity typically means retention in the stomach from about 4, 6, 8 or at times 10 hours and up to about 18 hours. It is however noted that in accordance with the invention, retention of the GRDA is not observed after more than 48 hours after administration, and preferably not after 24 hours.

The therapeutic effect achieved by the delivery of the macromolecule may be a local as well as a systemic therapeutic effect.

The term “local effect” denotes a therapeutic effect at the area (tissue or organ) of release of the macromolecule from the GRDA, i.e. within and bordering the GI tract. The macromolecule, having some degree of affinity to a target within the GI tract, preferably within the stomach or the small intestine binds to such a target leading to a therapeutic effect. For example, the macromolecule may have affinity to a receptor or antigen presented on the gastric lumen. Further, as an example, the macromolecule may be an inhibitor of phospholipase A2 localized at the gastrointestinal lumen thereby effective against phospholipase related conditions. A local effect may also be induced in all types of cells lining the GIT, as a result of the direct contact of the GRDA (e.g. adhesion) with those cells and not via the blood circulation.

In another example, the macromolecule is an antigen i.e., recombinant cholera toxin B-subunit (rCTB) against Helicobacter pylori that is lining within the gastric mucosa. The delivery of the antigen can locally suppress H. pylori proliferation.

In yet another example is the macromolecule a gastric or pancreatic lipase that degraded food lipids in the stomach as part of the digestion process. Local delivery of gastric lipase from the GRDA in the stomach will enable optimal availability of the enzyme in the gastric compartment.

The term “systemic effect” denotes the delivery of the macromolecule throughout the body via the transport of the macromolecule across the GI lumen into the blood stream. Macromolucles having molecular weight greater then 1000 are not effectively absorbed through the GI lumen (J. G. Russell-Jones, Carrier-mediated Transport, Oral Drug Delivery, in “Encyclopedia of Controlled Drug Delivery” 173, 175, E Mathiowitz ed. 1999). Various approaches to improve the oral absorption of drugs are under investigation (G L Amidon and H J Lee, Ann. Rev. Pharmac. Tox. 34, 321-241, 1994; M Goldberg and I Gomez-Orellana Nature Reviews Drug Discovery 2, 289-295, 2003; N N. Salama et al., JPET Fast Forward, Sep. 24, 2004). One attitude to improve the oral absorption of drugs may be to reversibly loosen the intestinal tight junctions so as to enhance their para-cellular transport and increase oral absorption. Absorption enhancers are capable of improving the transport/absorption of low bioavailable drugs. Some absorption enhancers specifically loosen tight junctions and enhance para-cellular permeability. Another approach to enhance absorption of macromolecules in the GIT is to modify them chemically, so as to make them more hydrophobic or amphiphilic (U.S. Pat. No. 6,656,922). Chemical modifications include for example the coupling of the macromolecules to linear aliphatic chain, pegilation, bile acids such as deoxycholic acid or glycocholic acid, cholesterol, alkanoic acids. Moreover, macromolecules may also be coupled to moieties which are recognized by specific transporters, thus allowing transporter-assisted absorption.

Thus, for example, the incorporation within the GRDA of the invention also absorption enhancers, either in the same polymeric layer or in a separate layer of the GRDA, or the modification (lipophilization or the like) of the macromolecule, may facilitate the systemic delivery of macromolecules carried by the GRDA. The enhancer can be co-released with the macromolecule with the same rate or in a different rate, according the needs of the specific application.

Additionally, the GRDA may also include adjuvant that enhance stability of the macromolecule once release into the GIT or that enhances the biological/therapeutic activity of the macromolecule. An example for such adjuvant can be protease inhibitor that inhibits photolytic enzymes. Inhibition of such enzymes enables the accumulation of the macromolecule in an intact form in the lumen and thus allowing an increase in the volume of the macromolecule to be available for intestinal absorption.

The GRDA of the invention may further comprise an anti-adhering material applied to at least a portion of the outer surfaces' of the device, so as to prevent sticking of the folded layers, and thus facilitate unfolding of the device once released from the delivery system and wetted.

The anti-adhering material may be such material as known to those versed in the art. Examples include, without being limited thereto, pharmaceutically acceptable celluloses, cellulose derivatives, silicates, glyceryl esters of fatty acids and others, or water repelling agents, i.e. simethicone, dimeticone, cyclomethicone and others. A preferred anti-adhering material comprises microcrystalline cellulose.

The GRDA of the invention may also comprise plasticizers. Examples of plasticizers include, without being limited thereto, citrate esters, phthalate esters, dibutyl sebacate, diacetylated monoglycerides, glycerin, glycerin derivatives (such as triacetin), polyethylene glycols, propylene glycol, sorbitol, or a combination of such plasticizers.

Further, the GRDA of the invention may comprise fillers. The filler may be starch, glucose, lactose, an inorganic salt, a carbonate, bicarbonate, a sulfate, a nitrate, a silicate, an alkali metal phosphate, an oxide, or a combination thereof.

In addition to the mentioned composition, the device may comprise lubricants, and other pharmaceutically acceptable processing adjutants, as known in the art.

Notwithstanding the above and in accordance with one embodiment, at least one layer or one enforcing strip of the device comprises a swellable polymer (hydrogel) to facilitate the unfolding of the device. The enveloping layers may comprise a polymer blend that swells in gastric fluid. Typically the weight of this layer in simulated gastric fluid is 100% to 400%, more preferable the weight increase increases 150% to 250% of its dry weight. The swelling causes significant expanding in the length (elongation) of the enveloping layer, of about 10 to 50%, more preferably of 20% to 30% in length.

As appreciated, while the invention is described above with reference to the macromolecule-containing GRDA, it is to be understood that also encompassed within the present invention is a method of preparing the GRDA for delivery of such macromolecules as well as a method of delivery of macromolecules to a subject by the GRDA.

Specifically, the method for preparing a GRDA for delivery of a macromolecule comprises (i) assembling a multi-layered device comprising a macromolecule-containing compartment bordered by enveloping layers, at least one enveloping layer is made of a polymeric film comprising at least one aperture or a polymeric composition comprising a material which dissolves upon contact with gastric fluid to form at least one aperture, and one or more enforcing strips, the enveloping layer adapted to release, upon unfolding in gastric fluids, the macromolecule from the macromolecule-containing compartment via the at least one aperture in the enveloping layer; (ii) folding said device; and (iii) introducing or combining the folded device with a delivery system.

As defined above, the material is a physiologically acceptable substance and may be a soluble polymer, as soluble acid salt and/or a soluble particle, preferably combined with a non-soluble polymer, such that upon contact with gastric fluid, pores or channels are formed in the layer comprising the non-soluble polymer. Examples include, without being limited thereto, hydroxyethylcellulose, polyethylene glycol (PEG) (MW>20,000), NaCl, CaCO₂, Cocoa butter, etc.

The invention also provides a method for the delivery of macromolecules to a subject's stomach, comprising oral administration of the GRDA of the invention to the subject, preferably by swallowing.

The delivery of the GRDA of the invention is preferably for the treatment of a pathological condition.

Thus, there is also provided a method of treating a pathological condition with the macromolecule acting as the active principle, the method comprises oral administering to a subject in need an amount of the GRDA of the invention, the amount being sufficient to obtain a therapeutic effect in said subject.

The terms “treating” or “treatment”, and the like are used herein to refer to obtaining a desired pharmacological and physiological effect. The effect may be prophylactic in terms of preventing or partially preventing a disease, symptom or pathological condition and/or may be therapeutic in terms of a partial or complete cure of a disease, condition, symptom or adverse effect attributed to a pathological condition. Thus, “treatment” covers any treatment of a disease in a mammal, particularly a human, and includes: (a) preventing a pathological condition from occurring in an individual which may be predisposed to develop a pathological condition but has not yet been diagnosed as having it, i.e., causing the clinical symptoms of a pathological condition not to develop in a subject that may be predisposed to develop the condition but does not yet experience or display symptoms of the condition; (b) inhibiting, i.e., arresting or reducing the development of the pathological condition or its clinical symptoms; or (c) relieving symptoms associating with the pathological condition.

The term “pathological condition” used herein denotes any condition which requires improving the well-being of the subject the delivery of a biologically functional and active macromolecule, the latter being as defined hereinbefore. This includes, inter alia, a condition selected from inflammation and autoimmune disorders, parasitism (e.g. malaria), bacterial, viral or fungal infection, cardiac disorders (e.g. arrhythmia), coagulation disorders, depression, diabetics, epilepsy, migraine, cancer, immune disorders, hormonal disorders, psychiatric conditions, gastrointestinal tract disorders, nutritional disorders, and many others, as known in the art.

According with one preferred embodiment of the invention, the condition is a “condition of the GI tract” (used interchangeably with the term “GI pathological condition”). This term denotes any condition of the GI tract, preferably the stomach or the small intestine, which is associated with an abnormality of the GI tract. This includes a disorder or disease where the primary abnormality of the GI tract is an altered physiological function (the way the body works) such as in the case of irritable bowel syndrome (IBS) and dyspepsia (which are the most common functional GI disorders), as well as structural disorders (having an identifiable structural or biochemical cause, such as in the case of GI polyps, cancer, ulcer etc.).

The following is a non-limiting list of GI pathological conditions that may be treated by the use of the GRDA of the invention:

Stomach-origin anomalies, such as, without being limited thereto, Gastroparesis, by local delivery of the peptide hormone CCK; Gastritis, by local delivery of anti inflammatory drugs; Gastroenteritis (viral or bacterial), by local delivery of antibiotics such as spectracef; Gastric ulcer (e.g. peptic ulcer disease), by local delivery of antacids, mucosal protective agents; Gastric cancer, by local chemotherapy.

Intestinal-origin anomalies, such as, without being limited thereto, Irritable Bowel Syndrome (IBS), GI bleeding, GI portal hypertension (viewed by the appearance of varices), all the three anomalies may be treated with local delivery of somatostatin-28 or its metabolic stable analogs; Colitis, by local therapy with drugs such as Mesalamin; GI cancer, by local delivery of chemotherapy, Carcinoid, by local delivery of therapeutic macromolecules; Inflammatory bowel disease (IBD), by local delivery of anti inflammatory agents; GI obstructions, by local delivery of mucosal protective agent such as mucin; metabolic diseases associated with excess or deficient secretion of gut hormones such as gastrin, motilin, somatostatin, secretin, vasoactive intestinal peptide (VIP), galanin, geralin, and enzymes such as amylase, lipase, pepsins, chymotrypsin and trypsin, by local delivery of therapeutic agents such as hormone receptors agonists or antagonists, hormone releasing or anti secretion agents, enzymes and their inhibitors, co-factors or substrates.

As appreciated by those versed in the art, the GRDA of the invention may be utilized for the delivery of macromolecules for systemic treatment. There are various conditions which may be treated by systemic delivery of macromolecules, including, for example only, Osteoporosis, by delivery of calcitonin; Female infertility, by delivery of suitable hormones; Immunodeficiency, by delivery of suitable growth factors, as well as other endocrine system-related conditions.

As will be shown in the following examples, PTH was efficiently released from the GRDA of the invention. Thus, in accordance with one embodiment, the macromolecule is preferably PTH for the treatment of osteoporosis.

The amount of macromolecule in the GRDA effective to achieve a desired therapeutic result, i.e. treatment of a pathological condition may be varied or adjusted widely depending upon the particular application, the release profile of the macromolecule from the GRDA, the potency of the particular macromolecule, the formulation/composition of the macromolecule in the macromolecule-containing compartment, and the desired concentration at the treated site. The effective amount is typically determined in appropriately designed clinical trials (dose range studies) and the person versed in the art will know how to properly conduct such trials in order to determine the effective amount. As generally known, an effective amount depends on a variety of factors including the affinity of the macromolecule to a target site, the selection of polymers forming the delivery device, the distribution profile of the macromolecule within the body after being released from the device and transported via the GI lumen, a variety of pharmacological parameters such as half life in the body, on undesired side effects, if any, and on other factors such as age and gender of the treated subject, etc.

The GRDA of the invention may be administered over an extended period of time in a single daily dose or on each consecutive day. The treatment period will generally have a length proportional to the length of the disease process and the specific GRDA effectiveness and the patient species being treated, all as being appreciated by those versed in the art.

As used in the specification and claims, the forms “a”, “an” and “the” include singular as well as plural references unless the context clearly dictates otherwise. For example, the term “a macromolecule” includes one or more, of the same or different macromolecules.

Further, as used herein, the term “comprising” is intended to mean that the layers of the device include the recited elements, but not excluding others which may be optional in the designed of the GRDA, such as plasticizers, fillers an the like. The term “consisting essentially of” is used to define layers that include the recited elements but exclude other elements that may have an essential significance effect on the release or lack of release of the macromolecule from the device. For example, a device where the matrix consists essentially of soluble polymer(s) will not include or include only insignificant amounts (amounts that will have an insignificant effect on the release of the macromolecule from the device) of polymers that prevent the dissolution of the matrix in the gastric fluid, such as enteric polymers. “Consisting of” shall thus mean excluding more than trace elements of other elements. Embodiments defined by each of these transition terms are within the scope of this invention.

Further, all numerical values, e.g. when referring the amounts or ranges of the elements constituting the device's components, are approximations which are varied (+) or (−) by up to 20%, at times by up to 10% of from the stated values. It is to be understood, even if not always explicitly stated that all numerical designations are preceded by the term “about”.

### Specific Examples

To avoid degradation of macromolecules over time by enzymes such as peptidases, all lab-ware in contact with the macromolecules were sterile. Sterile water was used to make the solutions, and all solutions were filtered through 0.2 micron filter.

Formulation of α-Melanocyte-Stimulating Hormone (α-MSH) in gastro-retentive delivery assembly (GRDA)

### Materials

α-MSH (Molecular weight of 1664.9, Calbiochem, Germany) was received as a lyophilized powder of its TFA salt. The peptide was made up in 1% acetic acid.

The concentration of α-MSH in various samples was analyzed by HPLC (Gemini (Phenomenex) 5 μC18, 250×4.6; mobile phase—0.1% Trifluoroacetic acid/Acetonotrile:0.1% Trifluoroacetic acid/Water, gradient elution, 1 mL/min; 100 μL injection, UV PDA detection at 280 nm).

### α-MSH GRDAs

The α-MSH GRDAs exemplified herein (designated GRDAs 1 to 3) were composed of three layers, a core containing a matrix accommodating the peptide; polymer strips (in the shape of a frame) of enforcing polymeric composition affixed to the core matrix, and two enveloping layers each covering one side of the matrix affixed with the strips, the enveloped layers comprising cross-linked hydrolyzed gelatin.

The layers were affixed by applying (by brush or spray) ethanol as an adhesion-inducing substance. The laminated, essentially flat assembly was sprayed with ethanol and powdered with microcrystalline cellulose (Avicel, FMC BioPolymers) on both external faces. The powdered laminate was then folded (in an accordion like manner) and enclosed into a hard gelatin capsule (E00, Capsugel).

The GRDAs were of oval-like shape, 45 mm long by 24 mm wide (at its widest point) before folding into an E00 hard gelatin capsule.

In all GRDAs described below, strips of enforcing polymeric composition were prepared by casting a solution consisting of Eudragit L100, (Degussa), ethylcellulose N100 (Hercules) and triacetin (Merck) in ethanol.

The enveloping layers were prepared from a solution consisting of enzymatically hydrolyzed gelatin (average molecular weight 10,000-12,000, Byco C, Croda), Eudragit S (Degussa) and glycerin in a mixture of ethanol-water (1:1). Glutaraldehyde (Merck), diluted in the same solvent was added whilst mixing before casting for cross linking and evaporation.

### GRDA 1

α-MSH was formulated to obtain a peptide-carrying film (matrix) using the components presented in Table 1.

TABLE 1
α-MSH carrying film forming formulation

| Component | Percentage (%) |
|---|---|
| α-MSH | 0.049 |
| Glycerine | 1.45 |
| Gelatine | 4.85 |
| Acetic acid 1% in water | 49.971 |
| Water | 43.68 |

The formulation was formed into a film by casting in a purpose-made mould that had cavities of single drug reservoir area, a polymeric solution comprising glycerine and gelatine in ethanol. To the thus formed polymeric solution cc-MSH dissolved in 1% acetic acid was added. The resulting mixture was dried under vacuum to obtain a film of the peptide-carrying matrix.

Polymeric strips comprising Eudragit L100 (48%), Ethylcellulose N100 (19.2%), triacetin (28.8%) were affixed to the peptide-carrying film, and two enveloping layers comprising enzymatically hydrolyzed gelatin (Byco C) crosslinked with glutaraldehyde (43%), Eudragit S (27%), potassium phosphate (2%), sodium hydroxide (1%) and glycerin (18%) were attached to each side of the film affixed with the strips.

### GRDA 2

α-MSH was formulated to obtain a peptide-carrying film using the components presented in Table 1.

TABLE 2
α-MSH carrying film forming formulation

| Component | Percentage (%) |
|---|---|
| α-MSH | 0.049 |
| Glycerine | 0.23 |
| Hydroxypropyl cellulose (Klucel EF) | 4.53 |
| Acetic acid 1% in water | 49.991 |
| Water | 45.2 |

The formulation was formed into a film by casting in a purpose-made mould that had cavities of single drug reservoir area, a polymeric solution comprising glycerine and hydroxypropyl cellulose in ethanol. To the thus formed polymeric solution a-MSH dissolved in 1% acetic acid was added. The resulting mixture was dried under vacuum to obtain a film of the peptide-carrying matrix.

Polymeric strips comprising Eudragit L100 (48%), Ethylcellulose N100 (19.2%), triacetin (28.8%) were affixed to the peptide-carrying film, and two enveloping layers comprising crosslinked enzymatically hydrolyzed gelatin (Byco E) (43%), Eudragit S (27%), potassium phosphate (2%), sodium hydroxide (1%) and glycerin (18%) were attached to each side of the film affixed with the strips.

### GRDA 3

Commercially available gelatine sheets (Merck, cat #104072 , food grade) were used as the basis of this formulation. The pre-cut sheets were soaked in the solution containing the α-MSH solution and dried under vacuum. This formulation was assembled as described above. However, one of the external shield was perforated twice with either punches of 0.7 mm diameter or 1.5 mm diameter.

### Formulation of Parathyroid Hormone (PTH 1-34) in Gastro-Retentive Delivery Assembly (GRDA)

### Materials

PTH 1-34 (molecular weight of 4117.8, UCB Bioproducts, USA) was received as a lyophilized powder. The peptide was made up in 1% acetic acid. The stability of the peptide in 1% acid solution was confirmed by incubating it for 24 hr at 37° C.

### Stability Assay

The stability of PTH 1-34 in various buffer solutions over 24 hr at 37° C. was evaluated to allow selection of a suitable buffer for in vitro release experiments. The lyophilized peptide (0.1 mg) was dissolved in each buffer solution that was tested. The concentration of PTH 1-34 in samples withdrawn from the solution at 0, 4, 8, and 24 hours were analyzed by HPLC (Gemini (Phenomenex) 5 μ C18, 250×4.6; mobile phase˜0.1% Trifluoroacetic acid/Acetonotrile:0.1% Trifluoroacetic acid/Water, gradient elution, 1 mL/min; 100 μL injection, UV PDA detection at 214).

### PTH 1-34 GRDAs

The PTH 1-34 GRDAs exemplified herein (GRDAs 4 to 7) are composed of three layers, a core containing a matrix accommodating the peptide; polymer strips (in a frame shape) of enforcing polymeric composition affixed to the core matrix, and two enveloping layers each covering one side of the matrix affixed with the strips, the enveloped layers comprising cross-linked hydrolyzed gelatin.

The layers were affixed by applying (by brush or spray) ethanol as an adhesion-inducing substance. The laminated, essentially flat assembly was sprayed with ethanol and powdered with microcrystalline cellulose (Avicel, FMC BioPolymers) on both external faces. The powdered laminate was then folded (in an accordion like manner) and enclosed into a hard gelatin capsule (E00, Capsugel).

The GRDAs were of oval shape, 45 mm long by 24 mm wide (at its widest point) before folding into an E00 hard gelatin capsule.

In all GRDAs described below, strips of enforcing polymeric composition were prepared by casting a solution consisting of Eudragit L100, (Degussa), ethylcellulose N100 (Hercules) and triacetin (Merck) in ethanol.

The enveloping layers were prepared from a solution consisting of enzymatically hydrolyzed gelatin (average molecular weight 10,000-12,000, Byco C, Croda), Eudragit S (Degussa) and glycerin in a mixture of ethanol-water (1:1). Glutaraldehyde (Merck), diluted in the same solvent was added whilst mixing before casting for cross linking and evaporation.

### GRDA 4

PTH 1-34 was formulated to obtain a peptide-carrying film using the components presented in Table 3.

TABLE 3
PTH 1-34 carrying film forming formulation

| Component | Percentage (%) |
|---|---|
| PTH 1-34 | 0.07 |
| Glycerine | 0.34 |
| Hydroxypropyl cellulose (Klucel EF) | 6.42 |
| Acetic acid 1% in water | 49.96 |
| Ethanol 96% | 43.21 |

The formulation was formed into a film by casting in a purpose-made mould that had cavities of single drug reservoir area, a polymeric solution comprising glycerine and hydroxypropyl cellulose in ethanol. To the thus formed polymeric solution PTH 1-34 dissolved in 1% acetic acid was added. The resulting mixture was dried under vacuum to obtain a peptide-carrying film.

Polymeric strips comprising Eudragit L100 (48%), Ethylcellulose N100 (19.2%), triacetin (28.8%) were affixed to the peptide-carrying film, and two enveloping layers comprising crosslinked enzymatically hydrolyzed gelatin (Byco E) (43%), Eudragit S (27%), potassium phosphate (2%), sodium hydroxide (1%) and glycerin (18%) were attached to each side of the film affixed with the strips.

### GRDA 5

The same components of GRDA 4 were utilized for GRDA 5; however in this case, one of the enveloping layers was punched (1, 2 or 3 punches) to create holes of 1.5 mm in diameter.

### GRDA 6

PTH 1-34 was formulated to obtain a peptide-carrying film using the components presented in Table 4:

TABLE 4
PTH 1-34 carrying film forming formulation

| Component | Percentage (%) |
|---|---|
| PTH 1-34 | 0.14 |
| Glycerine | 4.04 |
| Gelatin bloom 257 | 13.46 |
| Acetic acid 1% in water | 28.53 |
| Water | 53.83 |

The gelatin was allowed to dissolve and swell in water for 1 hour at 37° C., the temperature was then raised to 70° C. for 0.5 hour, glycerol was added and the solution mixed for 30 min with a magnetic stirrer. Gelatin (500 μl) and PTH 1-34 solution (200 μl in 1% acetic acid) were cast together in the mould. Casting into the tray was done on pretreated with simethicone (1 ml of 1% simethicone solution in ethylacetate per tray, drying 1 hour at 35° C.), under a nitrogen stream. Drying was carried out overnight under vacuum.

The final GRDA comprising the polymeric strips and enveloping layers was then prepared as described in assembly 2.

### GRDA 7

PTH 1-34 was formulated to obtain a peptide-carrying film using the components presented in Table 5.

TABLE 5
PTH 1-34 carrying film forming formulation

| Component | Percentage (%) |
|---|---|
| PTH 1-34 | 0.20 |
| Glycerine | 0.28 |
| Hydroxypropyl cellulose (Klucel GF) | 5.27 |
| Sterile water | 44.35 |
| Acetic acid 1% in water | 49.90 |

The hydroxypropyl cellulose polymer was dispersed in water at a temperature between 45° C. and 55° C. Peptide solution (the peptide dissolved in 1% acetic acid) was cast onto the tray, followed by the addition of the polymeric dispersion. The mixture was allowed to set for 2 hours, and then dried under vacuum overnight. The resulting peptide-carrying film was then assembled with the additional components of the assembly, as described in GRDA 5.

Formation of Film for Enveloping Layer with Macro-Pores in Gastro-Retentive Delivery Assembly (GRDA 8)

The following GRDA 8 is designed to provide an enveloping layer comprising apertures In order to create assemblies consisting of macro-pores in the outer enveloping layer (pores in the order of 100-200 μm) by the use of a conventional a freeze-dry procedure [Hae-Won Kim et al. J Biomed Mater Res A.; 72(2):136-45 (2005)].

### Materials

Sodium hydroxide-99 % AR was purchased from -BIOLAB; hydrolyzed gelatin (Byco C) EurPh, was purchased from Croda Chemicals; potassium phosphate, Dibasic-USP and glycerin-USP were purchased from J. T. Baker; Eudragit S-USP was purchased from Degussa; ethanol-USP/BP/EP-Pharmco products; sterile water was purchased from Teva Medical Ltd.

### Method

The enveloping layers are prepared using the components presented in Table 6.

TABLE 6
Enveloping layer formulation

| Component | Percentage (%) |
|---|---|
| Hydrolyzed gelatin (Byco C) | 46 |
| Eudragit S | 28.5 |
| Glycerin | 21.9 |
| NaOH | 1.46 |
| K₂HPO₄ | 2.19 |

The enveloping layer comprising apertures are prepared by first adding hydrolyzed gelatin (Byco C, 6.3 g) to a solution of 3 g glycerin and 28 ml water and stirring with an overhead stirrer for 1 hour at 37° C. Then, ethanol (23 g) is added gradually (drop-wise) to the gelatin/glycerin solution.

In a separate vessel, 3.9 g of Eudragit S is dispersed in 17 ml water. To the Eudragit S dispersion, a solution of 0.2 g NaOH and 0.3 g K₂HPO₄ prepared in 12 g Ethanol is added drop-wise. The mixture is stirred at 37° C. until Eudragit S is completely dissolved, after which, the mixture is added to the gelatin/glycerin solution The resulting mixture is then cast on Teflon-coated plate of 26×40 cm and the plate is manipulated in accordance with the following [Hae-Won Kim et al. J Biomed Mater Res A.; 72(2):136-45 (2005)]:

The plate is transferred promptly into a freezer and kept at −60° C. for 24 hours and then freeze-dried for another 72 hours;
The plate is air dried for 8 hours in a stove at 37° C. and relative humidity of 30%;
The plate is placed in a sealed container saturated with glutardialdehyde vapor for 3 days at 37° C. to obtain cross-linking of the hydrolyzed gelatin;
The plate is dried at ambient conditions for 8 hours.

As a result, a film is formed having pores created in the size range of between 100-200 μm in diameter. This size range is sufficient to allow the release of macromolecules.

### In Vitro Release Tests for the Different GRDAs

Release of the peptides from the different assemblies was examined by shaking the capsule containing the assembly in 100 mL buffer (KCl/HCl buffer pH=2.2) at 37° C.

### Results

### GRDA 1

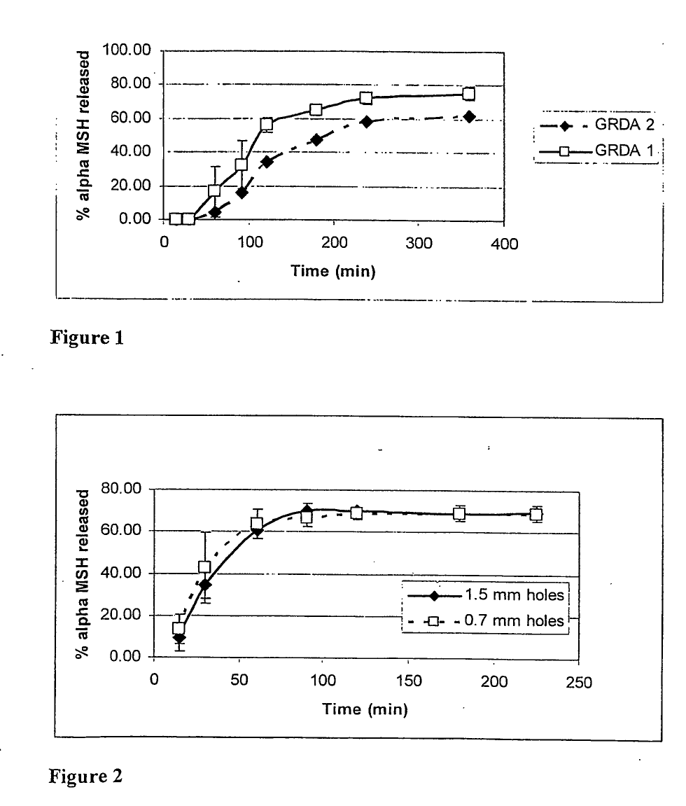

Samples were withdrawn from the system containing GRDA 1 at time periods up to 6.0 hours. It was found that after 4 hours 60% of the peptide was released into the buffer. The results are shown in FIG. 1.

### GRDA 2

Samples were withdrawn from the system containing GRDA 2 at time periods up to 6.0 hours. It was found that after 4 hours 70% of the peptide was released into the buffer. The results are shown in FIG. 1.

### GRDA 3

Samples were withdrawn from the system containing GRDA 3 at time periods up to 6.0 hours. It was found that after 4 hours 70% of the peptide was released into the buffer. The results are shown in FIG. 2. The size of the holes in the external shield did not affect the release rate of the peptide, indicating that passage across the shield membrane is not the rate determining step in this GRDA.

### GRDA 4

Samples were withdrawn from the system containing GRDA 4 at time periods up to 6.5 hours. No PTH 1-34 was detected in the solution after 6.5 hours.

### GRDA 5

Samples were withdrawn from the system containing GRDA 5 at time periods up to 6.0 hours. It was found that after 6 hours 75% of the peptide was released into the buffer.

### GRDA 6

Samples were withdrawn from the system containing GRDA 6 at time periods up to 6.0 hours. It was found that after 6 hours 75% of the peptide was released into the buffer. GRDA 7

Samples were withdrawn from the system containing GRDA 7 at time periods up to 10.0 hours. It was found that after 10 hours 80% of the peptide was released into the buffer.

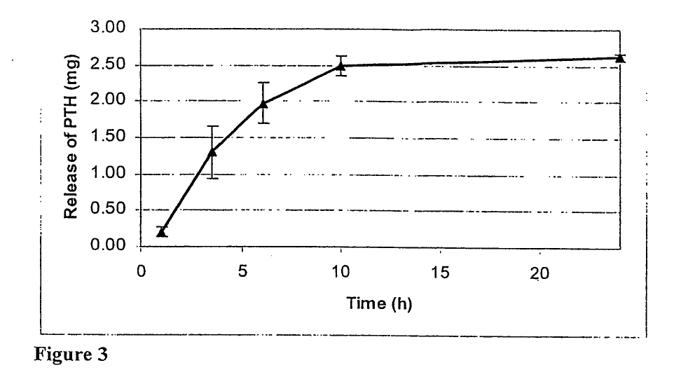

The release profile of GRDA 7 is shown in FIG. 3.

### Stability Assay

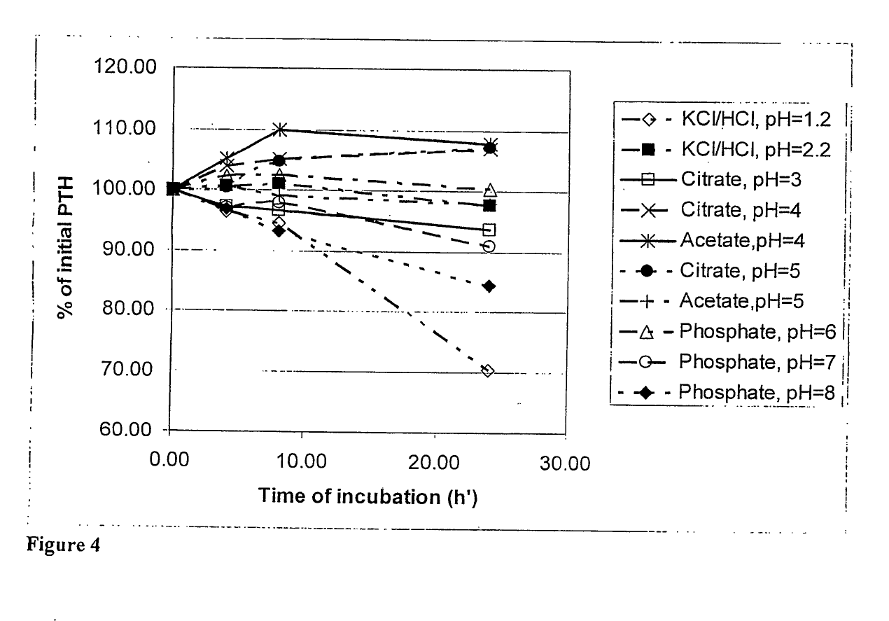

It was found that PTH 1-34 is stable over a wide range of pH values. The results of the stability study in various buffer solutions are summarized in FIG. 4. The peptide showed decreased stability at pH>7 and at pH=1.2. Thus, for further in vitro release tests, a KCl/Cl buffer pH=2.2 was selected. It is noted that the peptide is stable at this pH over 24 hr.

The above results show that for macromolecules there is a need to provide (or induce) apertures in the cross-linked gelatin containing enveloping layer in order to enable release of the macromolecule from the GRDA. It is believed that this requirement will be relevant for any macromolecule having a molecular weight of above 2000 Da.

1-31. (canceled)

32. A gastro-retentive delivery assembly (GRDA) comprising a folded multi-layered device comprising a macromolecule-containing compartment bordered by enveloping layers and optionally, one or more enforcing strips, the device being adapted to unfold when in a subject's stomach, whereupon unfolding, the macromolecule is released from said device via at least one aperture in an enveloping layer.

33. The GRDA of claim 32, wherein said macromolecule has a molecular weight of above about 1,800 Da.

34. The GRDA of claim 33, wherein said macromolecule has a molecular weight of above about 2,000 Da.

35. The GRDA of claim 34, wherein said macromolecule has a molecular weight of above about 3,000 Da.

36. The GRDA of claim 35, wherein said macromolecule has a molecular weight of above about 4,000 Da.

37. The GRDA of claim 32, wherein said macromolecule is essentially stable in gastric content.

38. The GRDA of claim 32, wherein said macromolecule is selected from carbohydrate, a nucleic acid molecule, an amino acid molecule, a lipid, a vitamin or vitamin analogue, a lipoprotein, a nucleoprotein, a lipopolysaccharide, a glycoprotein, a pegylated protein or pegylated polypeptide.

39. The GRDA of claim 38, wherein said macromolecule is a peptide, a polypeptide, a protein or a peptidomimetic macromolecule.

40. The GRDA of claim 32, wherein said macromolecule is a biologically active macromolecule.

41. The GRDA of claim 32, comprising two enveloping layers sandwiching the at least one macromolecule-containing compartment.

42. The GRDA of claim 32, wherein said at least one aperture in the enveloping layer is formed by mechanical puncturing of the enveloping layer, by applying laser beams onto said enveloping layer, or the at least one aperture is formed in said layer during production.

43. The GRDA of claim 32, wherein said at least one aperture in the enveloping layer is formed following administration of the GRDA to a subject in need.

44. The GRDA of claim 43, wherein at least one enveloping layer comprises a polymer, acid salts or particles soluble in gastric fluid, or a combination of same, whereupon contact with gastric fluid said polymer, salts and/or particles are dissolved, thereby forming a plurality of pores or apertures in the enveloping layer.

45. The GRDA of claim 32, folded within a delivery system.

46. The GRDA of claim 45, wherein said delivery system is a capsule.

47. The GRDA of claim 32, wherein said macromolecule is parathyroid hormone (PTH).

48. A method for gastroretentive delivery of macromolecules to a subject's stomach, the method comprising oral administration to said subject of the GRDA of claim 32.

49. A method for preparing a GRDA for delivery of a macromolecule comprising:
(i) assembling a multi-layered device comprising a macromolecule-containing compartment bordered by enveloping layers, at least one of said enveloping layers is made of a film comprising at least one aperture or a polymeric composition comprising a material which dissolves upon contact with gastric fluid to form at least one aperture and optionally, one or more enforcing strips, the device being adapted to unfold when in a subject's stomach, whereupon unfolding, the macromolecule is released from said device via the at least one aperture;
(ii) folding said device; and
(iii) introducing or combining the folded device with a delivery system.

50. The method of claim 49, wherein said at least one enveloping layer comprises a plurality of apertures.

51. The method of claim 49, wherein said aperture is formed by mechanical puncturing of the enveloping layer, by applying laser beams onto said enveloping layer, or said aperture is formed in said layer during production.

52. The method of claim 49, wherein said material is a polymer, acid salts or particles all of which being soluble in gastric fluid, or a combination of same, whereupon contact with gastric fluid said polymer, salts and/or particles are dissolved, thereby forming the plurality of apertures in the enveloping layer.

53. A method for treating a subject for a pathological condition, the method comprises providing said subject with a GRDA of claim 32.

54. The method of claim 53, comprising oral administration of said GRDA.

55. The method of claim 53, comprising administration of said GRDA once daily or on each consecutive day.

56. The method of claim 53, wherein said macromolecule is PTH.
